(19) United States
(12) Patent Application Publication
(10) Pub. No.: US 2026/0097193 A1
(43) Pub. Date: Apr. 9, 2026

(54) MULTIFUNCTION SKIN TREATMENT DEVICE AND METHOD

(71) Applicant: Shaul Rappaport, Los Angeles, CA (US)

(72) Inventor: Shaul Rappaport, Los Angeles, CA (US)

(21) Appl. No.: 18/905,988

(22) Filed: Oct. 3, 2024

(51) Int. Cl. A61M 37/00; A61N 1/36; A61N 5/06

(52) U.S. Cl. CPC A61M 37/0015; A61N 1/36014; A61N 5/0616; A61M 2037/0023; A61M 2037/0061; A61M 2205/051; A61M 2205/054; A61M 2210/04

(57) ABSTRACT

A disclosed skin treatment device and method are adapted for releasably attaching to the skin treatment device a plurality of different function heads to perform different treatment functions and releasably attaching a vial containing a skin serum. The method may further comprise detecting the function of the function head and adjusting the operation of the device. The method may further include detecting the vial of skin serum being fluidly connected to the skin treatment device. The method may also include activating the skin treatment device by a user and applying the function head to the user's skin and applying the serum to the user's skin in the area being treated by the function head.

### TECHNICAL FIELD

The invention generally relates to devices for treating and improving a person's skin and more particularly to a device and method providing multiple treatment functionality to treat and improve and person's skin and complexion.

### BACKGROUND

Various skin care treatment devices are well known. For example, some devices offer light treatment, while other devices offer electrical stimulation, or micro-needling. For example, it is known that light treatment can improve a person's skin complexion. Yet other devices offer serum application for a person's skin, particularly a person's facial skin. What is missing is a device that provides multiple functions thus allowing a user to improve their skin without changing devices. Therefore, there is an opportunity to improve skin treatment devices including a multifunctional device that overcomes the limitations and disadvantages that exist with current skin treatment devices.

### SUMMARY

The advantage of such a system would be to allow a user to coordinate the simultaneous use of skin treatments, including more than one type of skin treatment, and serums intended for the same outcome, thereby increasing the ultimate benefit of the treatment while lessening the time and effort required to apply said treatment.

One general aspect includes systems and methods for operating a multifunction skin treatment device. The systems and methods disclosed herein may include attaching, to the multifunction skin treatment device, a function head. The systems and methods disclosed herein may further include detecting, by a processor of the multifunction skin treatment device, via a first sensor, a skin treatment function of the function head and adjusting an operation of the multifunction skin treatment device based on a predetermined setting associated with the skin treatment function. The systems and methods disclosed herein may also include attaching, to the multifunction skin treatment device, a vial containing a skin serum. The systems and methods disclosed herein may further include detecting, by the processor, via a second sensor, the vial being fluidly connected to the multifunction skin treatment device. The systems and methods disclosed herein may also include activating, by a user, the skin treatment function and applying the multifunction skin treatment device to a skin surface. The systems and methods disclosed herein may further include applying, during the application of the function head, serum to the skin surface via the multifunction skin treatment device. Other embodiments of this aspect include corresponding computer systems, apparatus, and computer programs recorded on one or more computer storage devices, each configured to perform the actions of the methods.

Implementations may include one or more of the following features. The methods and systems where adjusting the operation of the multifunction skin treatment device based on the predetermined setting, may include adjusting a power and a frequency transmitted to the function head from the multifunction skin treatment device when activated. The methods and systems where the function head applies microneedling to the skin surface. The methods and systems where the function head applies infrared and red light to the skin surface. The methods and systems where the function head applies electrical muscle stimulation to the skin surface. The methods and systems where the application of serum is provided via an electronically controlled sprayer. The methods and systems where the skin serum is one of a plurality which may include a repairing serum, a hydrating serum, a toning serum, and a brightening serum. The methods and systems where the vial is attached to the multifunction skin treatment device via a watertight coupling. The methods and systems where the second sensor is one of a moisture sensor, a pressure sensor, and a flow sensor.

Implementations of the described techniques may include hardware, a method or process, or computer software on a computer-accessible medium.

### BRIEF DESCRIPTION OF THE DRAWINGS

Advantages of the present invention will be readily appreciated as the same becomes better understood by reference to the following detailed description when considered in connection with the accompanying drawings wherein:

FIG. 1 is a perspective view of a multifunction skin treatment device of a nonlimiting embodiment of the invention;

FIG. 2 is a front view of the multifunction skin treatment device of FIG. 1, without a treatment attachment or serum vial as seen in FIG. 1;

FIG. 3 is a back view of the device of FIG. 2;

FIG. 4 is a side view of the device of FIG. 2;

FIG. 5 is a top view of the device of FIG. 2;

FIG. 6 is a bottom view of the device of FIG. 2;

FIG. 7 is a side view of the device of FIG. 1, with a rotating needle treatment attachment;

FIG. 8 is a side view of the device of FIG. 1, with an electricity stimulation treatment attachment as seen in FIG. 1;

FIG. 9 is a side view of the device of FIG. 1 with a light treatment attachment;

FIG. 10 is top view of the device of FIG. 7;

FIG. 11 is a top view of the device of FIG. 8;

FIG. 12 is a top view of the device of FIG. 9;

FIGS. 13A-C are top, side and bottom views of the rotating needle treatment attachment;

FIGS. 14A-C are top, side and bottom views of the electricity stimulation treatment attachment;

FIGS. 15A-C are top, side and bottom views of the light treatment attachment;

FIGS. 16A-C are top, side and bottom views of the serum vial of the treatment device seen in FIG. 1;

FIG. 17 is a schematic drawing of an example operating environment of the multifunction skin treatment device; and

FIG. 18 is a flowchart showing an example process for operating the multifunction skin treatment device.

### DETAILED DESCRIPTION

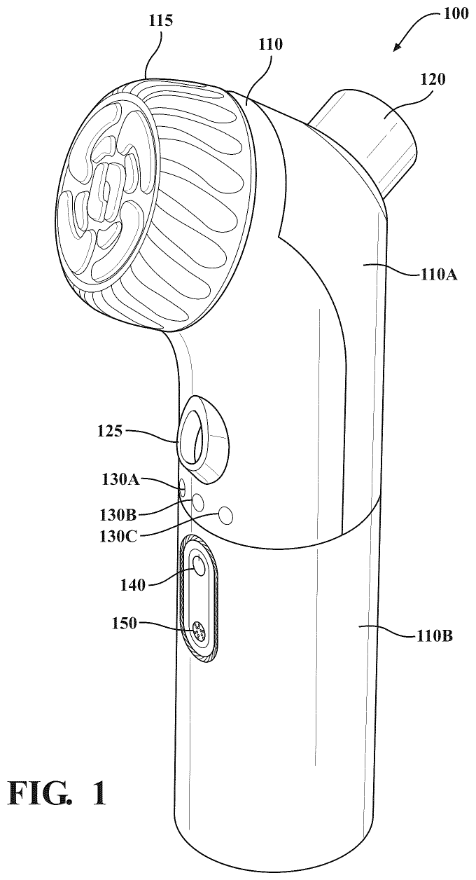

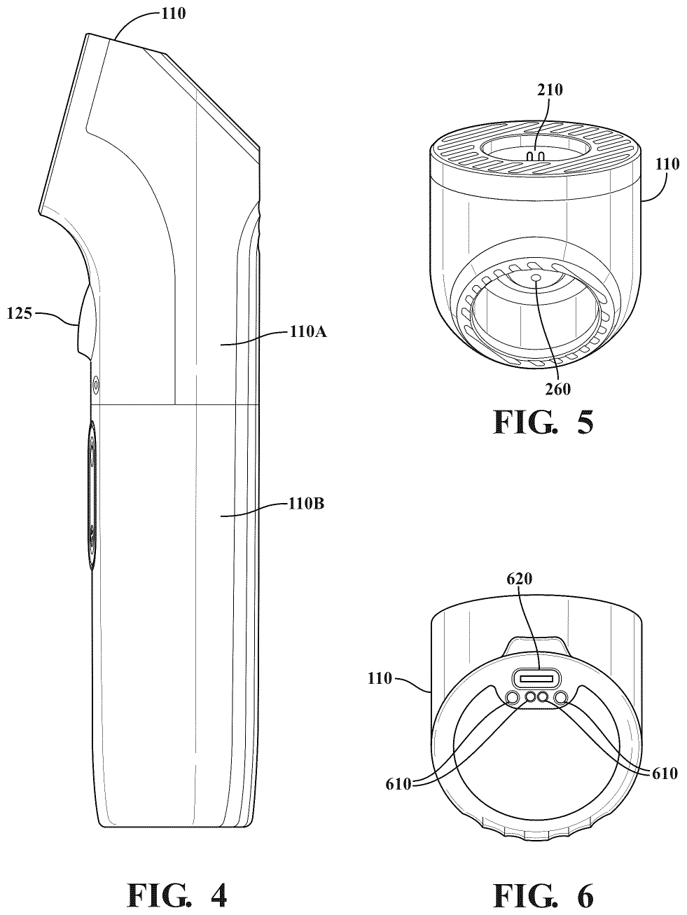

Referring to FIGS. 1-17, a multifunction skin treatment device 100 of the invention has application for use on a person's skin such as the person's facial skin. FIG. 1 provides a perspective view of an example embodiment of the multifunction skin treatment device 100. The multifunction skin treatment device 100 includes a body 110 that may be divided into a top portion 110A and a bottom portion 110B. The multifunction skin treatment device 100 also includes an interchangeable function head 115 of a plurality of available interchangeable function heads. In addition, the multifunction treatment device 100 may include an interchangeable vial 120 containing a substance to be applied to a user's skin.

In some embodiments, the multifunction skin treatment device 100 may include a spray nozzle 125 including a nozzle tip 125A positioned at the front of the multifunction skin treatment device, the angle at which the substance may be sprayed by the spray nozzle 125 may be variable. The spray nozzle 125 may be adjustable by a user or may include actuators which may automatically adjust the angle of the spray. Generally, the spray nozzle 125 may be positioned to directly spray the substance such as a liquid including a serum towards an area of a person's skin to be treated by the multifunction skin treatment device 100. The nozzle 125 and nozzle tip 125A may include two orifices, a first orifice is used to spray the serum and air thus breaking the serum down into a mist. The second orifice blows air to push the serum mist particles towards the user. The liquid can include any type of liquid suitable for spraying onto a person's skin including well known skin serums.

The multifunction skin treatment device 100 may further include indicator lights 130A, 130B, and 130C. In some embodiments, the multifunction skin treatment device 100 may be configured to determine which of the plurality of interchangeable function heads are installed onto the device and activate the indicator associated with that function head 115. For example, when a micro-needling function head is installed, the indicator light 130A may be activated to visually alert the user that the micro-needling function head has been installed. In some embodiments, the indicator light 130A may be associated with a micro-needling function head, the indicator light 130B may be associated with an electric muscle stimulation function head, and the indicator light 130C may be associated with a light therapy function head.

In some embodiments, when the function head 115 is removed, the associated indicator light is deactivated. In some embodiments, the color of the indicator may change based on the status of the installation of the function head 115. For example, green may indicate installed, red may indicate not installed, and orange may indicate that it is installed incorrectly.

The multifunction skin treatment device 100 may further comprise a power button/indicator 140 and a spray button/indicator 150. In some embodiments, the power button/indicator 140 may be configured to accept inputs from the user indicating a desire to activate and deactivate the multifunction skin treatment device 100 and to visually indicate whether the multifunction skin treatment device 100 is active or inactive. For example, a user may look to power button/indicator 140 and determine the multifunction skin treatment device 100 is deactivated, the user may then engage the power button/indicator 140 thereby activating the multifunction skin treatment device 100; activation of which may be indicated by the power button/indicator 140 being lit up.

Similarly, the spray button/indicator 150 may be configured to accept inputs from the user indicating a desire to activate and deactivate the spray function of the multifunction skin treatment device 100 and may visually indicate whether the spray function is active or inactive. For example, the user may engage the spray button/indicator 150 when the multifunction skin treatment device 100 is activated, and a mist may emit from the sprayer until the spray button/indicator 150 is again engaged to deactivate the spray function. In some embodiments, the spray function may be activated for a predetermined amount of time with each press, where the predetermined time may be based on the type of serum detected in the skin serum vial 120.

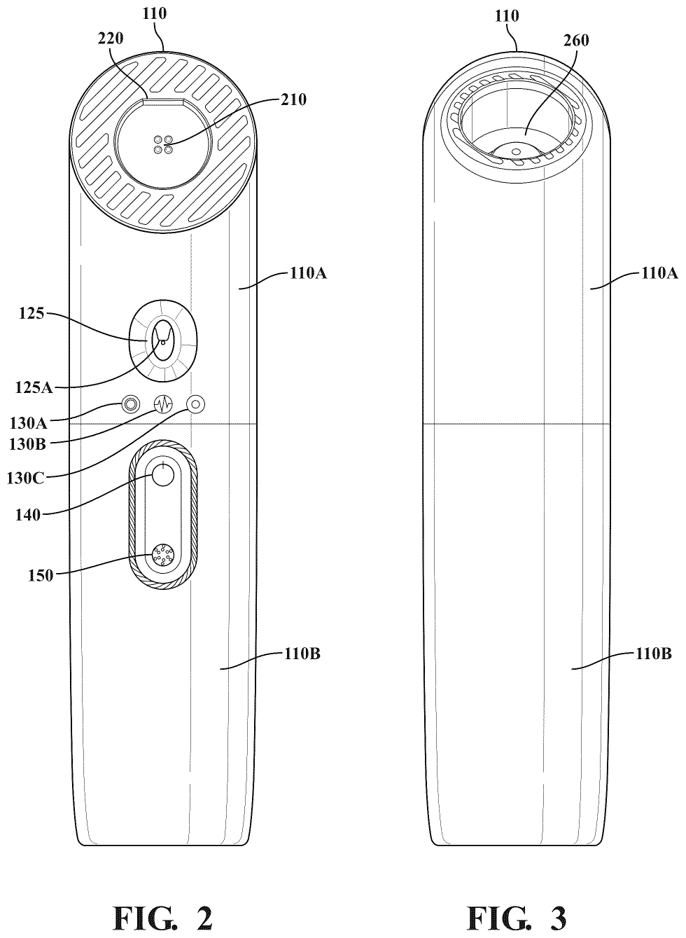

FIGS. 2 and 3 illustrate the body 110 multifunction skin treatment device 100 in an example embodiment having no function head installed and no vial of serum 120 inserted to better illustrate the contact points 210, a head alignment notch 220, and vial intake 260. The remainder of the multifunction skin treatment device 100 is the same as that illustrated in FIG. 1.

In some embodiments, the multifunction skin treatment device 100 may comprise the contact points 210 which may be configured to supply power with the function head when installed. For example, the contact points 210 may be formed from a conductive material such as copper, tungsten, silver, gold, platinum, and the like. In some embodiments, the function head 115 may have contact points configured to align with the contact points 210 when installed and provide power by conducting electricity from the multifunction skin treatment device 100 to the function head 115.

In some embodiments, the multifunction skin treatment device 100 may include a head alignment notch 220 which may be configured to ensure the correct orientation of the function head 115 when installed. In some embodiments, the function head 115 may have a reciprocal protrusion (described in more detail below) that mates with the head alignment notch 220 only when the function head 115 has the correct orientation.

In some embodiments, the multifunction skin treatment device 100 may include the vial intake 260 for inserting and holding a serum vial 120. For example, a serum vial 120 may have a watertight coupling (not shown) that mates with the multifunction skin treatment device 100 and creates a watertight seal fluidly connecting the serum vial to the multifunction skin treatment device 100. In some embodiments, the serum vial 120 may be configured to remain in the vial intake 260 during the operation of the multifunction skin treatment device 100.

FIGS. 5 and 6 illustrate a side view and top view, respectively, of an example embodiment of the body 110 of multifunction skin treatment device 100 without the function head 115 installed and no serum vial 120 inserted into vial intake 260. The remainder of the body 110 multifunction skin treatment device 100 is the same as that illustrated in FIG. 1.

FIG. 6 illustrates an example embodiment of the multifunction skin treatment device from a bottom view showing a group of contact points 610 and a port input 620. The remainder of the multifunction skin treatment device 100 is the same as that illustrated in FIG. 1.

In some embodiments, the multifunction skin treatment device 100 may include the group of contact points 610 which align with a reciprocal group of contact points in a charging base (not shown) and, once coupled, may be configured to allow power to flow from the charging base to the multifunction skin treatment device 100. For example, the contact points 610 may be formed from a conductive material such as copper, tungsten, silver, gold, platinum, and the like to accommodate the flow of electricity. The charging base is of the type well known to a person of ordinary skill in the art.

In some embodiments, the multifunction skin treatment device 100 may include the port input 620, which may be configured to charge the device and communicate with an external computing device. For example, the port input 620 may be compatible with, but not limited to, micro-USB, USB type-A, USB type-B, USB type-C, mini-USB, APPLE LIGHTING, and the like.

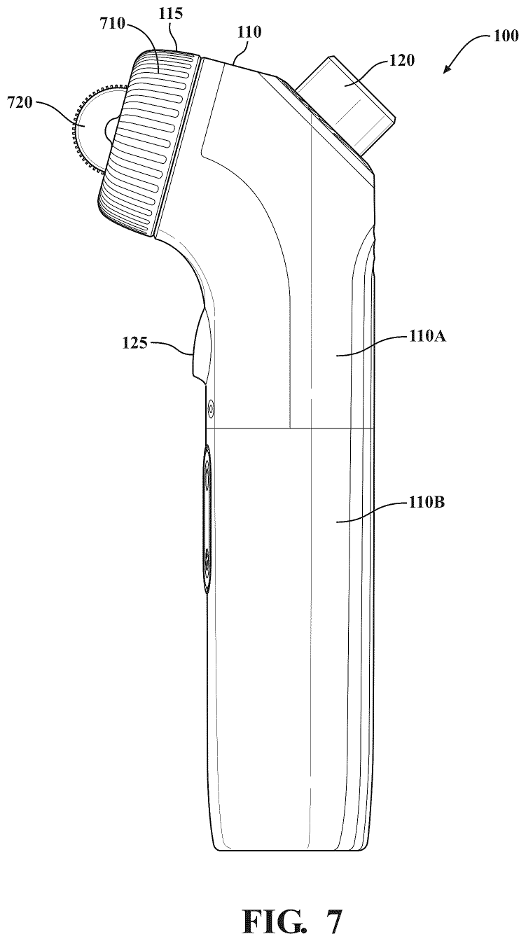

FIG. 7 illustrates the multifunction skin treatment device 100 in an example embodiment having a micro-needling 710 function head 115 installed and a serum vial 120 inserted from a side perspective. The remainder of the multifunction skin treatment device 100 is the same as that illustrated in FIG. 1.

In some embodiments, the micro-needling 710 function head 115 may comprise a micro-needling roller 720 having a cylindrical shape, the surface of which is covered with fine needles. The micro-needling roller 720 is rotationally supported by the micro-needling function head 710 so that in operation it will roll along a surface such as a person's facial skin surface while a user holds and moves the multifunction skin treatment device 100 while directing micro-needling roller 720 along the skin surface. Using the micro-needling roller 720 is believed to offer many benefits, the main one being collagen and elastin production which are the two building blocks of healthy skin, resulting in stronger, smoother skin. These and other benefits are well known. In some embodiments, the fine needles may be made from stainless steel, aluminum, titanium, or any other appropriate medical-grade steel.

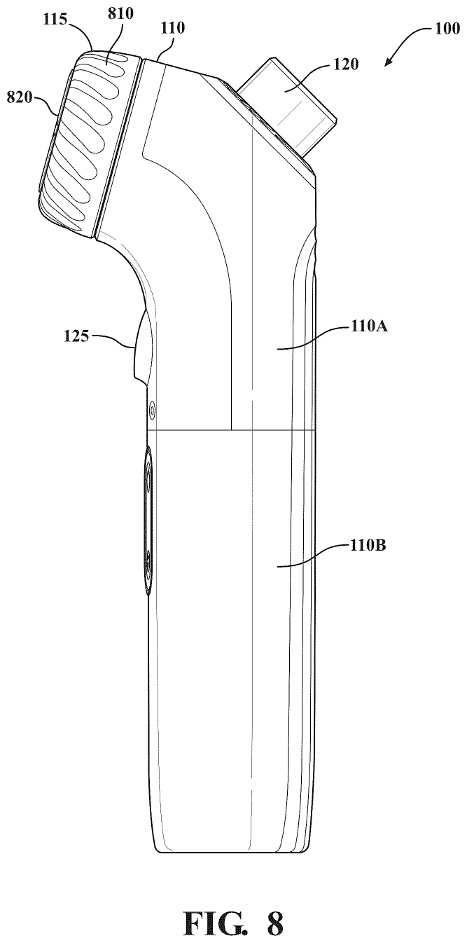

FIG. 8 illustrates the multifunction skin treatment device 100 in an example embodiment having an electrical muscle stimulation 810 function head 115 installed and a serum vial 120 inserted from a side perspective. The remainder of the multifunction skin treatment device 100 is the same as that illustrated in FIG. 1.

In some embodiments, the electrical muscle stimulation 810 function head 115 may comprise a group of electrode pads 820 which may be configured to apply an electrical current to the surface of the skin of a user. For example, the multifunction skin treatment device 100 may be configured to, via the group of electrode pads 820, apply electrical impulses to target the nerves and muscles of the user. Electrical stimulation is known to provide many benefits to skin and can be used to soften wrinkles and rejuvenate the skin.

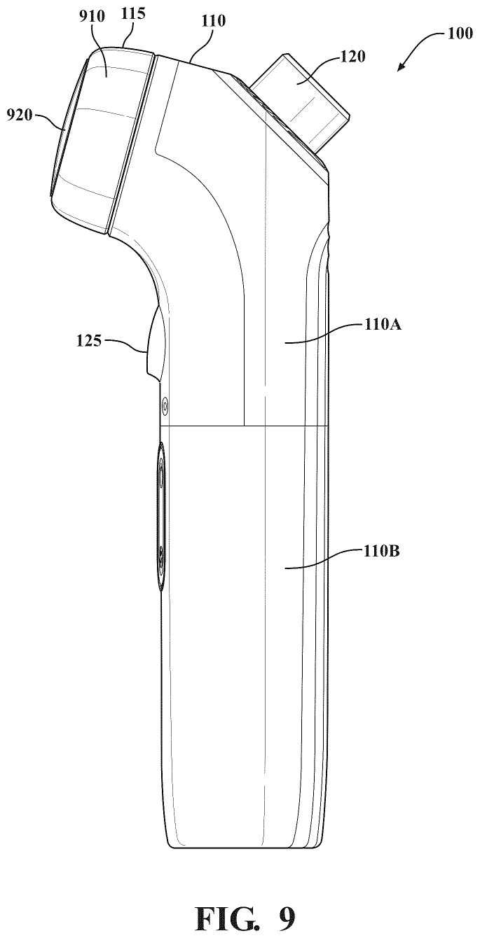

FIG. 9 illustrates the multifunction skin treatment device 100 in an example embodiment having a light therapy 910 function head 115 installed and a serum vial 120 inserted from a side perspective. The remainder of the multifunction skin treatment device 100 is the same as that illustrated in FIG. 1.

In some embodiments, the light therapy 910 function head 115 may comprise one or more light sources (e.g., LEDs, incandescent light bulbs, fluorescents, and the like) in order to produce light beneficial for the skin (e.g., red light, blue light, yellow light, infrared light, or some combination thereof). Light therapy is known to treat various skin conditions and concerns, such as acne, fine lines and psoriasis

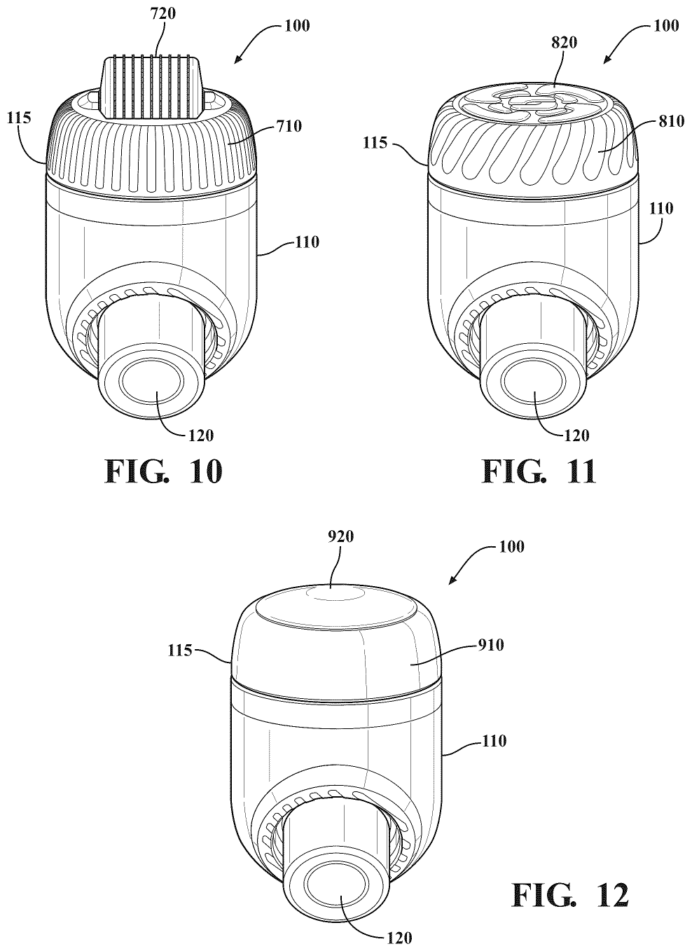

FIG. 10 illustrates a top-down view of an example embodiment of the multifunction skin treatment device 100 with the micro-needling 710 function head 115 installed and serum vial 120 inserted.

FIG. 11 illustrates a top-down view of an example embodiment of the multifunction skin treatment device 100 with the electrical muscle stimulation 810 function head 115 installed and serum vial 120 inserted.

FIG. 12 illustrates a top-down view of an example embodiment of the multifunction skin treatment device 100 with the light therapy 910 function head 115 installed and serum vial 120 inserted.

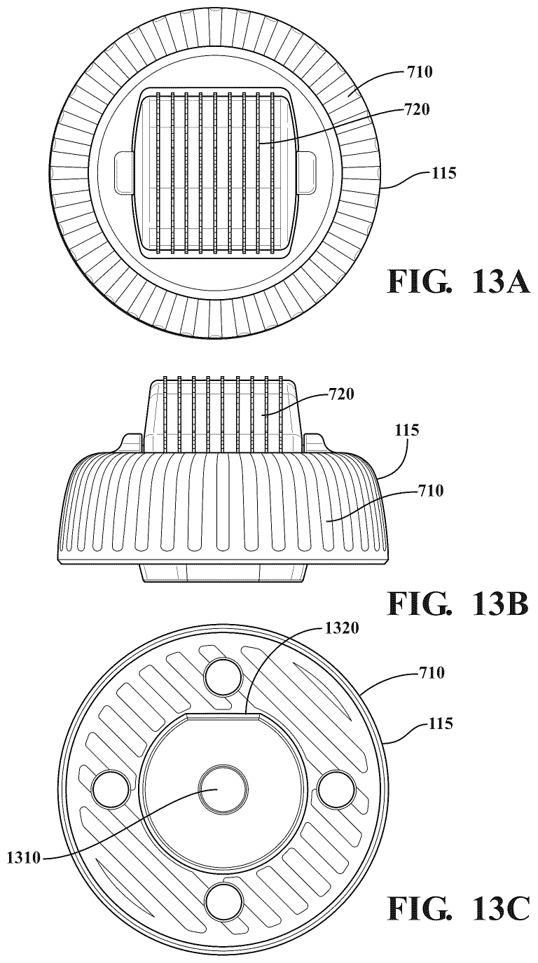

FIG. 13 illustrates multiple angles of the micro-needling 710 function head 115 independent of the multifunction skin treatment device 100. In some embodiments, the micro-needling function head 710 may further comprise an alignment protrusion 1320, which may mate with head alignment notch 220, thereby ensuring the correct orientation of the micro-needling function head 710 when installed.

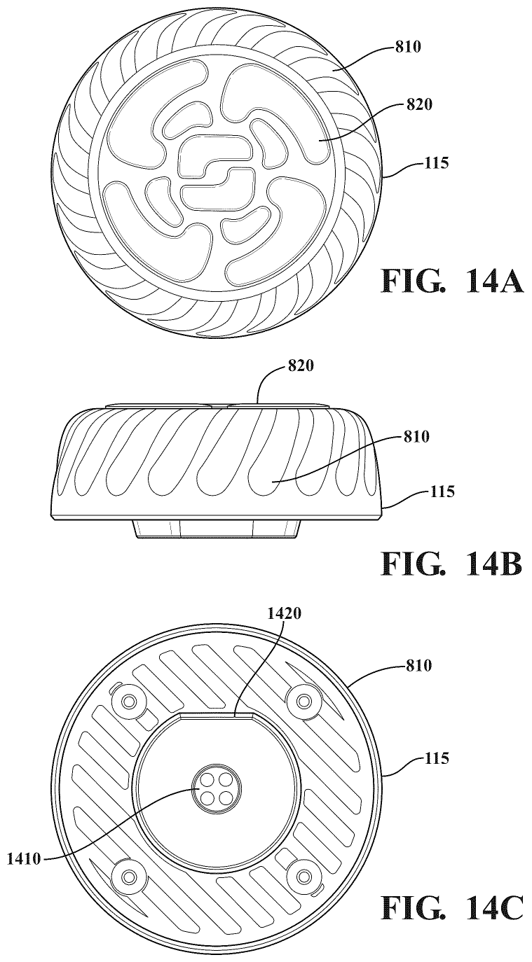

FIG. 14 illustrates multiple angles of the electrical muscle stimulation 810 function head 115 independent of the multifunction skin treatment device 100. In some embodiments, the electrical muscle stimulation f810 unction head 115 may further comprise an alignment protrusion 1420, which may mate with head alignment notch 220, ensuring the correct orientation of the electrical muscle stimulation 810 function head 115 when installed.

In some embodiments, the electrical muscle stimulation 810 function head 115 may further comprise a group of contact points 1410 configured to supply power with the electrical muscle stimulation 810 function head 115 when installed. For example, the group of contact points 1410 may be formed from a conductive material such as copper, tungsten, silver, gold, platinum, and the like. In some embodiments, the electrical muscle stimulation 810 function head 115 may have contact points configured to align with the contact points 210 when installed and provide power by conducting electricity from the multifunction skin treatment device 100 to the electrical muscle stimulation 810 function head 115.

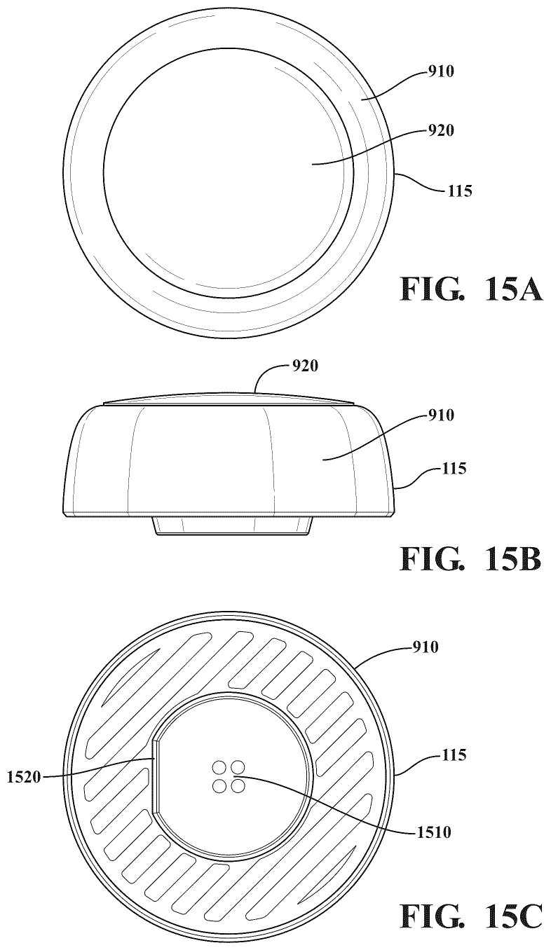

FIG. 15 illustrates multiple angles of the light therapy 910 function head 115 independent of the multifunction skin treatment device 100. In some embodiments, the light therapy 910 function head 115 may further comprise an alignment protrusion 1520, which may mate with head alignment notch 220, thereby ensuring the correct orientation of the light therapy 910 function head 115 when installed.

In some embodiments, the light therapy 910 function head 115 may further comprise a group of contact points 1510 configured to supply power with the light therapy head 910 when installed. For example, the group of contact points 1510 may be formed from a conductive material such as copper, tungsten, silver, gold, platinum, and the like. In some embodiments, the light therapy 910 function head115 may have contact points configured to align with the contact points 210 when installed and provide power by conducting electricity from the multifunction skin treatment device 100 to the light therapy 910 function head 115.

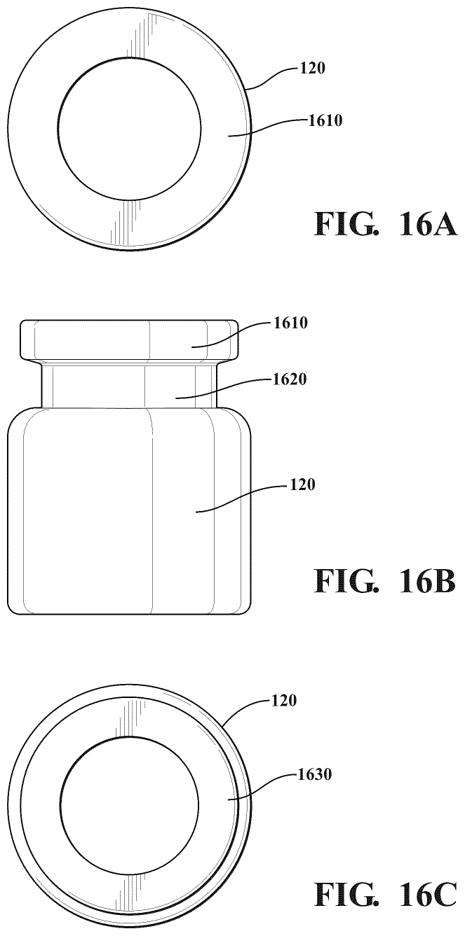

FIG. 16 illustrates multiple angles of the serum vial 120 independent of the multifunction skin treatment device 100. In some embodiments, the serum vial 120 may comprise a rim 1610, a neck 1620, and a bottom 1630. In some embodiments, the serum vial 120 may mate with a seal ring (not shown) that may sit between the serum vial 120 and the multifunction skin treatment device 100 ensuring a watertight seal. In some embodiments, the seal ring may comprise of a flexible watertight material (e.g., rubber, silicone, latex, PVC, foam, or some combination thereof).

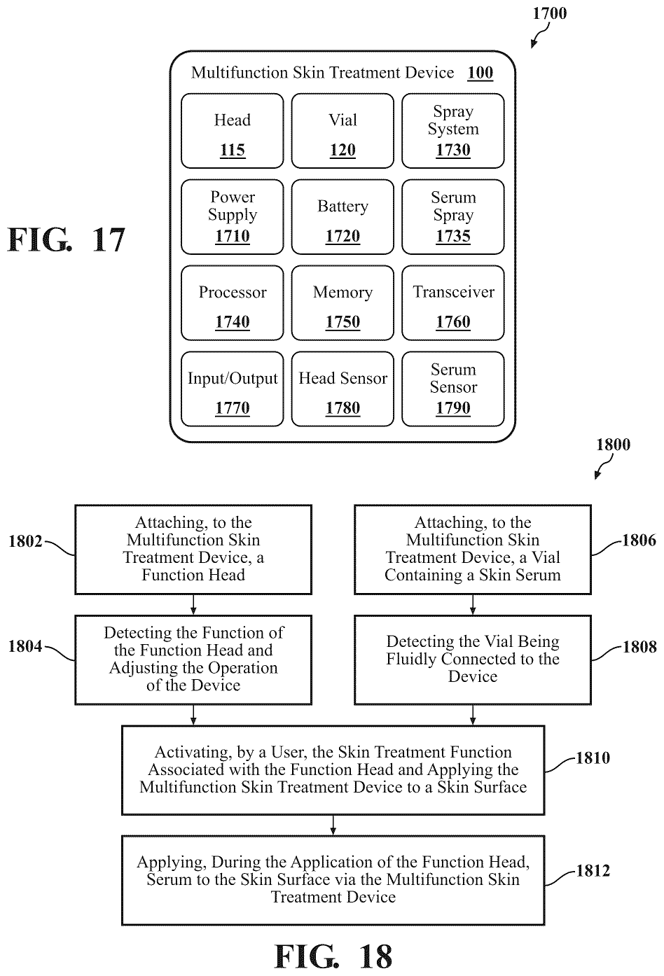

FIG. 17 is a block diagram illustrating an operating environment 1700 for managing the operation of the multifunction skin treatment device 100. The representation of the operating environment 1700 illustrates the components of the multifunction skin treatment device 100, some or all of which may be contained therein.

In general, the multifunction skin treatment device 100 includes a system of one or more computers configured to perform particular operations or actions by virtue of having software, firmware, hardware, or a combination of them installed on the system that, in operation, causes the system to perform the actions. One or more computer programs can be configured to perform particular operations or actions by virtue of including instructions that, when executed by a data processing apparatus, cause the apparatus to perform the actions.

As already described, the multifunction skin treatment device 100 may include a function head 100 and a vial 120. In some embodiments, the operating environment 1700 may include a power supply 1710, a battery 1720, and a processor 1740. In some embodiments, the processor 1740 may be communicatively linked to the battery 1720 and the power supply 1710 and may be configured to control the operation of the multifunction skin treatment device 100. For example, the processor 1740 may indicate to the power supply 1710 that the power from the battery 1720 must be converted to a specific level/frequency and direct where that power must be sent.

In some embodiments, the processor 1740 may further be communicatively coupled to a memory 1750, a transceiver 1760, an input-output interface 1770. In some embodiments, the memory 1750 may be a non-transitory computer-readable medium capable of storing instructions in a computer-readable format. For example, processor 1740 may detect a function head 115 attached to the multifunction skin treatment device 100 and access the memory 1750 for instructions related to operating the multifunction skin treatment device 100 when performing the function related to the detected function head, such as directing the correct power at the correct frequency from the battery 1720 to the function head 115.

In some embodiments, the input-output interface 1770 may be configured to accept inputs from a user and output information to the user. For example, the input-output interface 1770 may comprise the indicator lights 130A, 130B, and 130C, the power button/indicator 140, and the spray button/indicator 150. In some embodiments, the transceiver 1760 may send and receive communications from an external computing device. For example, the transceiver 1760 may communicate via Bluetooth, wireless fidelity (WIFI), infrared, cellular, wireless local area network (WLAN), microwave, near field communication (NFC), a global positioning system (GPS), radio, and any other appropriate form of wireless communication.

In some embodiments, the processor 1740 may further be communicatively coupled to a spray system 1730, a serum spray 1735, a head sensor 1780, and a serum sensor 1790. The spray system 1730 includes a pump or spray device as is well known to a person of ordinary skill in the art and adapted to spray or turn into a mist a liquid and direct the spray or mist to a surface such as a person's skin surface. In some embodiments, the serum spray 1730 sprays the serum from the serum vial 120 through the spray nozzle 125 and the nozzle tip 125A. In some embodiments, the spray system 1730 and serum spray 1730 may comprise an electronically controlled actuator and spray mechanism, an ultrasonic atomizer, an electric sprayer, or any appropriate mechanisms for spraying the serum.

In some embodiments, head sensor 1780 may identify which function head 115 is installed on the multifunction skin treatment device 100, based on which, the processor 1740 may alter the parameters of how the multifunction skin treatment device 100 is operating. For example, the head sensor 1780 may be configured to identify the function head 115 via the amount of resistance perceived from the contact points, whether or not there are contact points on the function head 115, or detect some signal output by the function head 115 (e.g., NFC, BLUETOOTH beacon, infrared, or any appropriate form of short-range communication).

In some embodiments, serum sensor 1790 may be configured to identify whether or not a serum vial 120 is installed on the multifunction skin treatment device 100 as well as which serum is in the serum vial 120, based on which, the processor 1740 may alter the parameters of how the multifunction skin treatment device 100 is operating. For example, the serum sensor 1790 may detect a serum with a higher viscosity than a previous serum; based on that information, the processor 1740 may direct the serum spray 1730 to operate at a lower rate of output. In some embodiments, the serum sensor 1790 may comprise a moisture sensor, a viscosity sensor, a color sensor, a pH sensor, a conductivity sensor, a dissolved oxygen sensor, an ion sensor, and any sensor appropriate for determining the characteristics of a liquid.

FIG. 18 illustrates an example process 1800 for operating the multifunction skin treatment device 100. Any block of process 1800 may be executed by the processor 1740. The blocks of process 1800 may be performed in sequence, out of sequence, in parallel, with extra blocks, and with some blocks missing.

At block 1802, the process 1800 may comprise attaching a function head to the multifunction skin treatment device 100. For example, the multifunction skin treatment device 100 may have no function head installed, and a user may select any one of the micro-needling 710 function head 115, the electrical muscle stimulation 810 function head 115, and the light therapy 910 function head 115 to install on the contact points 210 and the head alignment notch 220. In some embodiments, the installation of the function head 115 further comprises aligning the head alignment notch 220 with a counterpart alignment protrusion on the function head 115. In some embodiments, the function head 115 may be installed by being screwed into place, held in place magnetically, clicked into place via one or more fasteners, with VELCRO hook and loop fasteners, a reusable adhesive, or any appropriate way of holding the function head in place.

At block 1804, the process 1800 may further comprise detecting, via the head sensor 1780, a skin treatment function of the function head 115 and adjusting an operation of the multifunction skin treatment device 100 based on a predetermined setting associated with the skin treatment function. In some embodiments, the detection of the function head 115 may occur via the processor 1740, and the predetermined settings associated with skin treatment functions may be stored in the memory 1750. For example, the user may install the light therapy head 910, and the head sensor 1780 may detect that the light therapy function is indicated, which may cause the processor to access the instruction in the memory 1750 associated with the light therapy function and alter the operation of the multifunction skin treatment device 100 based on said instructions.

At block 1806, the process 1800 may also comprise attaching, to the multifunction skin treatment device 100, the serum vial 120 in the vial intake 260. For example, the skin serum may comprise one of a repairing serum, a hydrating serum, a toning serum, and a brightening serum. In some embodiments, a seal ring may be used to create a water-tight seal between the serum vial 120 and the multifunction skin treatment device 100. In some embodiments, the seal ring may comprise a semi-solid material (e.g., rubber, silicone, latex, PVC, foam, or some combination thereof).

At block 1808, the process 1800 may further comprise detecting, by the processor 1740, via the serum sensor 1790, the vial being fluidly connected to the body 110 of the multifunction skin treatment device 100. For example, the serum sensor 1790 may detect moisture from the serum, the detection of which is reported to the processor 1740. In another example, the serum sensor 1790 may detect a serum with a higher viscosity than a previous serum; based on that information, the processor 1740 may direct the serum spray 1730 to operate at a lower rate of output. In some embodiments, the serum sensor 1790 may comprise a moisture sensor, a viscosity sensor, a color sensor, a pH sensor, a conductivity sensor, a dissolved oxygen sensor, an ion sensor, and any sensor appropriate for determining the characteristics of a liquid.

At block 1810, the process 1800 may further comprise activating, by a user, the skin treatment function and applying the multifunction skin treatment device 100 to the skin surface. For example, the user may activate the multifunction skin treatment device 100 via the power button/indicator 140. In some embodiments, the user may direct the surface of the function head to the surface of their skin to initiate skin treatment, where the angle also aligns the serum spray to mist the skin surface in an area proximate the treatment head 115.

At block 1812, the process 1800 may also comprise applying, during the application of the function head 115, serum to the skin surface via spray nozzle 125, including the nozzle tip 125A. For example, the user may activate the serum spray function via spray button/indicator 150 which initiates a spray of serum from the nozzle tip 125A of the spray nozzle 125.

In operation of the multifunction skin treatment device 100 a user would select and put in place a function head 115 depending on the desired skin treatment to be received. Once put in place, the multifunction skin treatment device 100 will recognize the type of head attached and adjust a treatment function best suited for the selected head. The user then has the option of selecting and putting in place a serum vial 120 containing a selected serum. Next, the user can press the spray button/indicator 150 and activate the spray or misting of the serum on the skin in and around the area being treated by the function head 115.

The disclosed multifunction skin treatment device 100 offers a combination of benefits including the synergistic benefit of combining multiple options for treatment heads with a serum that is intended to be most effective with a particular treatment head. Examples include a serum best suited for being applied while using the light therapy function with the light therapy head 910. Another example is combining a serum best suited for use with the light or micro-needling roller head. The multifunction skin treatment device 100 offers the benefit of a customized and controlled coordinated treatment. The disclosed system further offers the benefits of virtually unlimited combinations of treatment heads and serums.

### Clauses

Clause 1. A method for operating a multifunction skin treatment device, comprising:
attaching, to the multifunction skin treatment device, a function head, wherein the function head is releasably attachable to the multifunction skin treatment device and replaceable with more than one type of the function head;
detecting, by a processor of the multifunction skin treatment device, via a first sensor, a skin treatment function of the function head and adjusting an operation of the multifunction skin treatment device based on a predetermined setting associated with the skin treatment function;
attaching, to the multifunction skin treatment device, a vial containing a liquid adapted to be sprayed from the multifunction skin treatment device onto a skin surface wherein the vial is releasably attachable to the multifunction skin treatment device and replaceable;
activating, by a user, the skin treatment function and applying the multifunction skin treatment device to a skin surface; and
applying, during the application of the function head, the skin serum to the skin surface via the multifunction skin treatment device.

Clause 2. The method of any of the preceding clauses, further comprising, detecting, by the processor, via a second sensor, the vial being fluidly connected to the multifunction skin treatment device.

Clause 3. The method of any of the preceding clauses, wherein the liquid is a skin serum.

Clause 4. The method of any of the preceding clauses, wherein adjusting the operation of the multifunction skin treatment device based on the predetermined setting, comprises adjusting a power and a frequency transmitted to the function head from the multifunction skin treatment device when activated.

Clause 5. The method of any of the preceding clauses, wherein the function head applies microneedling to the skin surface.

Clause 6. The method of any of the preceding clauses, wherein the function head applies at least one of infrared and red light to the skin surface.

Clause 7. The method of any of the preceding clauses, wherein the function head applies electrical muscle stimulation to the skin surface.

Clause 8. The method of any of the preceding clauses, wherein the second sensor detects a type of the liquid associated with the vial containing the liquid and adjusts a rate of application of the liquid by the multifunction skin treatment device.

Clause 9. The method of any of the preceding clauses, wherein the application of the liquid is provided via an electronically controlled sprayer.

Clause 10. The method of any of the preceding clauses, wherein the liquid is a skin serum comprising one of a repairing serum, a hydrating serum, a toning serum, and a brightening serum.

Clause 11. The method of any of the preceding clauses, wherein the vial is attached to the multifunction skin treatment device via a watertight coupling.

Clause 12. The method of any of the preceding clauses, wherein the second sensor is one of a moisture sensor, a pressure sensor, and a flow sensor.

Clause 13. A multifunction skin treatment device comprising:
a processor and a non-transitory computer-readable medium having instructions, that, when executed, cause the multifunction skin treatment device to:
detect, via a first sensor, installation of a function head and a skin treatment function of the function head and adjust an operation of the multifunction skin treatment device based on a predetermined setting associated with the skin treatment function;
activate, in response to a user input, the skin treatment function and apply the multifunction skin treatment device to a skin surface; and
apply, during the application of the function head, the skin serum to the skin surface via the multifunction skin treatment device.

Clause 14. The method of any of the preceding clauses, wherein the processor and the non-transitory computer-readable medium further cause the multifunction skin treatment device to detect, via a second sensor, a vial of a skin serum being fluidly connected to the multifunction skin treatment device.

Clause 15. The method of any of the preceding clauses, wherein the processor, when adjusting the operation of the multifunction skin treatment device based on the predetermined setting, adjusts a power and a frequency transmitted to the function head from the multifunction skin treatment device when activated.

Clause 16. The method of any of the preceding clauses, wherein the second sensor detects a type of serum associated with the vial containing the skin serum and adjusts a rate of application of the skin serum by the multifunction skin treatment device.

Clause 17. The method of any of the preceding clauses, wherein the application of serum is provided via an electronically controlled sprayer.

Clause 18. The method of any of the preceding clauses, wherein the vial is attached to the multifunction skin treatment device via a watertight coupling.

Clause 19. A multifunction skin treatment device, comprising:
a device body;
at least two treatment heads each providing a different skin treatment when releasably attached to the body;
at least one vial containing a serum adapted for treating the skin when releasably attached to the body;
wherein the body further comprises:
a treatment head receptor portion adapted to releasably receive one of the at least two treatment heads;
a vial receptor portion adapted to releasably receive the vial;
a sprayer in fluid communication with the vial receptor portion; and
a switch for activating the sprayer;
wherein disposed in the body is a processor and a non-transitory computer-readable medium having instructions, that, when executed, cause the multifunction skin treatment device to detect, via a first sensor, installation of one of the function heads and based on the type of function head adjust an operation of the multifunction skin treatment device based on a predetermined setting associated with a skin treatment function.

Clause 20. The method of any of the preceding clauses, wherein the processor and the non-transitory computer-readable medium further cause the multifunction skin treatment device to detect, via a second sensor, the vial being releasably attached to the device body.

Clause 21. The method of any of the preceding clauses, wherein the processor, when adjusting the operation of the multifunction skin treatment device based on the predetermined setting, adjusts a power and a frequency transmitted to the releasably attached function head.

Clause 22. The method of any of the preceding clauses, wherein the second sensor detects a type of serum associated with the vial containing a skin serum and adjusts a rate of application of the skin serum by the sprayer.

Clause 23. The method of any of the preceding clauses, wherein the serum is one of a plurality comprising a repairing serum, a hydrating serum, a toning serum, and a brightening serum.

Clause 24. The method of any of the preceding clauses, wherein the second sensor is one of a moisture sensor, a pressure sensor, and a flow sensor.

Several embodiments have been discussed in the foregoing description. However, the embodiments discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

What is claimed is:

1. A method for operating a multifunction skin treatment device, comprising:
attaching, to the multifunction skin treatment device, a function head, wherein the function head is releasably attachable to the multifunction skin treatment device and replaceable with more than one type of the function head;
detecting, by a processor of the multifunction skin treatment device, via a first sensor, a skin treatment function of the function head and adjusting an operation of the multifunction skin treatment device based on a predetermined setting associated with the skin treatment function;
attaching, to the multifunction skin treatment device, a vial containing a liquid adapted to be sprayed from the multifunction skin treatment device onto a skin surface wherein the vial is releasably attachable to the multifunction skin treatment device and replaceable;
activating, by a user, the skin treatment function and applying the multifunction skin treatment device to a skin surface; and
applying, during the application of the function head, the skin serum to the skin surface via the multifunction skin treatment device.

2. The method of claim 1, further comprising, detecting, by the processor, via a second sensor, the vial being fluidly connected to the multifunction skin treatment device.

3. The method of claim 1, wherein the liquid is a skin serum.

4. The method of claim 1, wherein adjusting the operation of the multifunction skin treatment device based on the predetermined setting, comprises adjusting a power and a frequency transmitted to the function head from the multifunction skin treatment device when activated.

5. The method of claim 1, wherein the function head applies microneedling to the skin surface.

6. The method of claim 1, wherein the function head applies at least one of infrared and red light to the skin surface.

7. The method of claim 1, wherein the function head applies electrical muscle stimulation to the skin surface.

8. The method of claim 2, wherein the second sensor detects a type of the liquid associated with the vial containing the liquid and adjusts a rate of application of the liquid by the multifunction skin treatment device.

9. The method of claim 1, wherein the application of the liquid is provided via an electronically controlled sprayer.

10. The method of claim 1, wherein the liquid is a skin serum comprising one of a repairing serum, a hydrating serum, a toning serum, and a brightening serum.

11. The method of claim 1, wherein the vial is attached to the multifunction skin treatment device via a watertight coupling.

12. The method of claim 2, wherein the second sensor is one of a moisture sensor, a pressure sensor, and a flow sensor.

13. A multifunction skin treatment device comprising:
a processor and a non-transitory computer-readable medium having instructions, that, when executed, cause the multifunction skin treatment device to:
detect, via a first sensor, installation of a function head and a skin treatment function of the function head and adjust an operation of the multifunction skin treatment device based on a predetermined setting associated with the skin treatment function;
activate, in response to a user input, the skin treatment function and apply the multifunction skin treatment device to a skin surface; and
apply, during the application of the function head, the skin serum to the skin surface via the multifunction skin treatment device.

14. The device of claim 13, wherein the processor and the non-transitory computer-readable medium further cause the multifunction skin treatment device to detect, via a second sensor, a vial of a skin serum being fluidly connected to the multifunction skin treatment device.

15. The device of claim 13, wherein the processor, when adjusting the operation of the multifunction skin treatment device based on the predetermined setting, adjusts a power and a frequency transmitted to the function head from the multifunction skin treatment device when activated.

16. The device of claim 14, wherein the second sensor detects a type of serum associated with the vial containing the skin serum and adjusts a rate of application of the skin serum by the multifunction skin treatment device.

17. The device of claim 13, wherein the application of serum is provided via an electronically controlled sprayer.

18. The device of claim 13, wherein the vial is attached to the multifunction skin treatment device via a watertight coupling.

19. A multifunction skin treatment device, comprising:
a device body;
at least two treatment heads each providing a different skin treatment when releasably attached to the body;
at least one vial containing a serum adapted for treating the skin when releasably attached to the body;
wherein the body further comprises:
a treatment head receptor portion adapted to releasably receive one of the at least two treatment heads;
a vial receptor portion adapted to releasably receive the vial;
a sprayer in fluid communication with the vial receptor portion; and
a switch for activating the sprayer;
wherein disposed in the body is a processor and a non-transitory computer-readable medium having instructions, that, when executed, cause the multifunction skin treatment device to detect, via a first sensor, installation of one of the function heads and based on the type of function head adjust an operation of the multifunction skin treatment device based on a predetermined setting associated with a skin treatment function.

20. The multifunction skin treatment device of claim 19, wherein the processor and the non-transitory computer-readable medium further cause the multifunction skin treatment device to detect, via a second sensor, the vial being releasably attached to the device body.

21. The multifunction skin treatment device of claim 19, wherein the processor, when adjusting the operation of the multifunction skin treatment device based on the predetermined setting, adjusts a power and a frequency transmitted to the releasably attached function head.

22. The system of claim 20, wherein the second sensor detects a type of serum associated with the vial containing a skin serum and adjusts a rate of application of the skin serum by the sprayer.

23. The system of claim 19, wherein the serum is one of a plurality comprising a repairing serum, a hydrating serum, a toning serum, and a brightening serum.

24. The system of claim 20, wherein the second sensor is one of a moisture sensor, a pressure sensor, and a flow sensor.
